(19) United States
(12) Patent Application Publication
(10) Pub. No.: US 2005/0075566 A1
(43) Pub. Date: Apr. 7, 2005

(54) Ultrasonice diagnosing apparatus

(75) Inventor: Yoshiaki Satoh, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/940,775

(22) Filed: Sep. 15, 2004

(30) Foreign Application Priority Data
Sep. 19, 2003 (JP) 2003-328210

(51) Int. Cl. A61B 8/00

(52) U.S. Cl. 600/443

(57) ABSTRACT

An ultrasonic diagnosing apparatus having a diagnosis support function for supporting an observational and quantitative diagnosis whether a tumor formed in a tissue of an object is benignant or malignant based on information on the object. The ultrasonic diagnosing apparatus includes: an image information generating unit for generating image information on an object based on ultrasonic echoes; an image information storage unit for storing the image information; a judgment criterion value setting unit for setting as a judgment criterion value a value to be used for judging an evaluation value on a tissue with respect to the image information; a judging unit for judging a state of the tissue by comparing the evaluation value with the judgment criterion value; and a display unit for displaying an ultrasonic image in an imaging mode and displaying a judgment result by the judging unit in a diagnosis support mode.

### BACKGROUND OF THE INVENTION

1. Field of the Invention

The present invention relates to an ultrasonic diagnosing apparatus for imaging organs within a living body etc. by transmitting and receiving ultrasonic waves so as to generate ultrasonic images to be used for diagnosis.

2. Description of a Related Art

In an ultrasonic diagnosing apparatus for medical application, normally, an ultrasonic probe including plural ultrasonic transducers having transmitting and receiving functions of ultrasonic waves is used. By using such ultrasonic probe, an object to be inspected is scanned by an ultrasonic beam formed by synthesizing the plural ultrasonic waves and the ultrasonic echoes reflected inside the object are received, and thereby, image information on tissues of the object is obtained based on the intensity of the ultrasonic echoes. Further, image information on movement of blood within the object is obtained based on frequency modulation information according to the Doppler effect included in the ultrasonic echoes.

As a related technology, in Japanese Patent Application Publication JP-A-6-254097, an ultrasonic diagnosing apparatus capable of realizing three-dimensional display in combination of a B-mode image and a CFM (color flow mapping) image is disclosed. According to the ultrasonic diagnosing apparatus, plural two-dimensional images are generated by performing projection processing on three-dimensional distribution information that has been obtained by distributing each image of outline images and blood stream images of the region of interest extracted from tissue information. Then, these two-dimensional images are continuously displayed in a predetermined order so that the cubic structure of the region of interest and the cubic structure of the blood stream image can be simultaneously observed due to a human visual property called motion parallax. For example, the relationship between a tumor and nutrient vessels thereof can be grasped.

However, in the case where the judgment whether the tumor is benignant or malignant is performed by using the ultrasonic diagnosing apparatus disclosed in JP-A-6-254097, a doctor empirically judges based on the relationship between the tumor and the nutrient vessels thereof. Therefore, the judgment largely depends on the experience and intuition of the doctor, and there has been a problem of lacking in objectivity and quantification.

By the way, in mammography CAD (computer-aided diagnosis), at the time of judgment whether the tumor is benignant or malignant, the judgment is made based on the complexity of the outline of the tumor or based on the degree of concentration of the blood vessels in the vicinity of the tumor. In the ultrasonic diagnosis, similarly, the observational and quantitative judgment as to whether the tumor is benignant or malignant based on the complexity of the outline of the tumor is being studied.

In Japanese Patent Application Publication JP-P2000-126182A, a tumor diagnosing method is disclosed in which a tumor (especially, breast tumor) region can be found with high accuracy from an ultrasonic three-dimensional image and the judgment of a malignant tumor can be automatically extracted with good reproducibility. According to the tumor diagnosing method, irregularities of the tumor surface is quantified by defining a parameter of an S/V ratio of a surface area S to a volume V of the (benignant or malignant) tumor extracted as a three-dimensional image by using a visualizing technology such as the ultrasonic diagnosing method. Thereby, interfaces between the tissues represented as the three dimensional image constituted by MRI images, ultrasonic images and so on of the living body are extracted so that a cancer tissue, especially, breast cancer tissue (breast malignant tumor) can be found from normal tissues.

Further, in Okuno et al., “Evaluation Doppler Ultrasonography for Breast Tumors: Study of Differential Diagnosis Using Arterial Waveform Pattern”, J Med Ultrasonics, 2003, Vol. 30, No. 3, P. 327-334, a study on breast tumors according to the ultrasonic Doppler method is described. According to the document, PI (pulsality index) and RI (resistance index) as indices of blood stream waveforms obtained in accordance with the ultrasonic Doppler method are both recognized as having a significant difference between a benignant tumor and a malignant tumor, but overlap largely. Accordingly, there described that, it is impossible to judge whether the tumor is a cancer or a benignant tumor determinably with respect to each case, but it is considered clinically effective to use them with the B-mode ultrasonic diagnosis complementarily.

However, an ultrasonic diagnosing apparatus having a diagnosis support function for practically enabling such diagnosis has not been proposed.

### SUMMARY OF THE INVENTION

The present invention has been achieved in view of the above-described problems. An object of the present invention is to provide an ultrasonic diagnosing apparatus having a diagnosis support function for supporting an observational and quantitative diagnosis as to whether a tumor formed in a tissue of an object to be inspected is benignant or malignant based on image information on the object.

In order to solve the above-described problems, an ultrasonic diagnosing apparatus according to the present invention includes: image information generating means for generating image information on an object to be inspected based on plural ultrasonic echoes obtained by scanning the object while sequentially transmitting plural ultrasonic beams from an ultrasonic probe; image information storage means for storing the image information generated by the image information generating means; judgment criterion value setting means for setting as a judgment criterion value at least one kind of value to be used for judging at least one kind of evaluation value on a tissue within the object with respect to the image information stored in the image information storage means; judging means for judging a state of the tissue by comparing the at least one kind of evaluation value on the tissue with the set judgment criterion value with respect to the image information as a target of judgment stored in the image information storage means; and display means for displaying an ultrasonic image based on the image information stored in the image information storage means in an imaging mode, and displaying a judgment result by the judging means in a diagnosis support mode.

According to the present invention, an observational and quantitative diagnosis as to whether a tumor formed in a tissue of an object to be inspected is benignant or malignant can be supported by setting one kind of judgment criterion value with respect to the image information stored in the image information storage means and then comparing at least one kind of evaluation value on the tissue with the one kind of judgment criterion value.

### BRIEF DESCRIPTION OF THE DRAWINGS

FIG. 1 is a block diagram showing the constitution of an ultrasonic diagnostic apparatus according to one embodiment of the present invention;

FIG. 2 is a flowchart showing the operation of the ultrasonic diagnosing apparatus as shown in FIG. 1 in a diagnosis support mode;

FIG. 3 shows an image formed by synthesizing a B-mode image and a Doppler image;

FIG. 4 shows set ROI;

FIG. 5 shows an extracted outline of a tumor;

FIG. 6 shows a measurement area at a distance “t” from the outline;

FIG. 7 shows relationship between an evaluation value “P” and the distance “t”;

FIG. 8 shows an image formed by synthesizing a graph representing the relationship between the evaluation value “P” and the distance “t” and a judgment criterion value setting field with the ultrasonic image;

FIG. 9 is an enlarged view of the judgment criterion value setting field as shown in FIG. 8; and

FIG. 10 is an image formed by synthesizing the judgment result with the B-mode image and the Doppler image.

### DESCRIPTION OF THE PREFERRED EMBODIMENTS

Hereinafter, a preferred embodiment of the present invention will be described in detail by referring to the drawings.

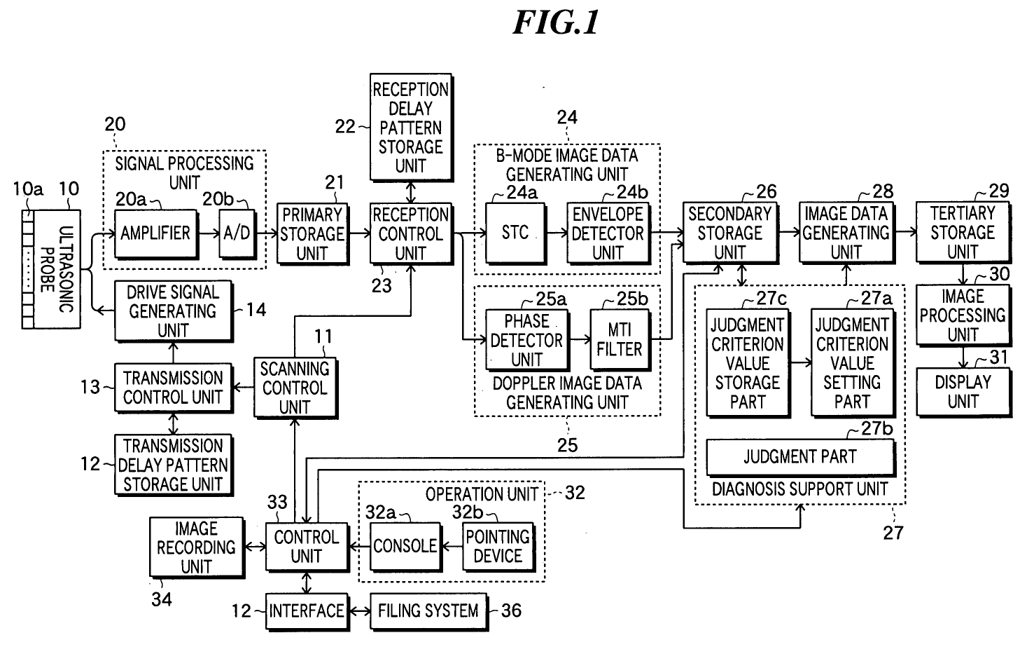

FIG. 1 is a block diagram showing the constitution of an ultrasonic diagnosing apparatus according to one embodiment of the present invention. The ultrasonic diagnostic apparatus according to the embodiment includes an ultrasonic probe 10, a scanning control unit 11, a transmission delay pattern storage unit 12, a transmission control unit 13 and a drive signal generating unit 14.

The ultrasonic probe 10 to be used by being abutted against an object to be inspected includes plural ultrasonic transducers 10a that form a one-dimensional or two-dimensional transducer array. These ultrasonic transducers 10a transmit ultrasonic beams based on applied drive signals, and receive ultrasonic echoes to output detection signals.

Each ultrasonic transducer is constituted by a vibrator in which electrodes are formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride), or the like. When a voltage is applied to the electrodes of the vibrator by generating a pulse or continuous wave electric signal, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves to generate electric signals. These electric signals are output as detection signals of ultrasonic waves.

Alternatively, as the ultrasonic transducers, plural kinds of elements of different ultrasonic conversion types may be used. For example, the above described vibrators are used as elements for transmitting ultrasonic waves and photo-detection type ultrasonic transducers are used as elements for receiving ultrasonic waves. The photo-detection type ultrasonic transducer is for detecting ultrasonic waves by converting ultrasonic signals into optical signals, and constituted by a Fabry-Perot resonator or fiber Bragg grating, for example.

The scanning control unit 11 sets the transmission direction of ultrasonic beams and the reception direction of ultrasonic echoes sequentially. The transmission delay pattern storage unit 12 has stored plural transmission delay patterns to be used when ultrasonic beams are formed.

The transmission control unit 13 selects a predetermined one of the plural delay patterns stored in the transmission delay pattern storage unit 12 in correspondence with the transmission direction set in the scanning control unit 11, and sets delay times to be provided to drive signals of the plural ultrasonic transducers 10a based on the pattern.

The drive signal generating unit 14 is constituted by, for example, plural pulsers corresponding to the plural ultrasonic transducers 10a, respectively. These pulsers generate drive signals based on the delay times set in the transmission control unit 13.

Further, the ultrasonic diagnostic apparatus according to the embodiment includes a signal processing unit 20, a primary storage unit 21, a reception delay pattern storage unit 22, a reception control unit 23, a B-mode image data generating unit 24, a Doppler image data generating unit 25, a secondary storage unit 26, a diagnosis support unit 27, an image data generating unit 28, a tertiary storage unit 29, an image processing unit 30 and a display unit 31.

The signal processing unit 20 includes plural amplifiers 20a and plural A/D converters 20b in correspondence with the plural ultrasonic transducers 10a. The detection signals output from the ultrasonic transducers 10a are amplified in the amplifiers 20a and the levels of these detection signals are matched with the input signal levels of the A/D converters 20b.

The analog signals outputted from the amplifiers 20a are converted into digital signals by the A/D converters 20b, respectively. As a sampling frequency of the A/D converter, at least about a tenfold frequency of that of the ultrasonic wave is required, and a 16-fold or more frequency of that of the ultrasonic wave is desirable. Further, as the resolving power of the A/D converter, resolving power of ten or more bits is desirable.

The primary storage unit 21 stores plural detection signals that have been converted into digital signals in the plural A/D converters 20b, respectively, in chronological order. The reception delay patterns to rage unit 22 has stored plural reception delay patterns to be used when reception focusing processing is performed on the plural detection signals outputted from the plural ultrasonic transducers 10a.

The reception control unit 23 performs reception focusing processing by selecting a predetermined one of the plural delay patterns stored in the reception delay pattern storage unit 22 in correspondence with the reception direction set in the scanning control unit 11, and providing delay times to the plural detection signals based on the pattern and adding the detection signals. By the reception focusing processing, sound ray data is formed in which the focus of the ultrasonic echo is narrowed. In addition, the reception focusing processing may be performed before the A/D conversion by the A/D converters 20b. The sound ray data formed by the reception control unit 23 is inputted to the B-mode image data generating unit 24 and the Doppler image data generating unit 25.

The B-mode image data generating unit 24 generates B-mode image data as cross sectional image information on a tissue within the object. The B-mode image data generating unit 24 includes an STC (sensitivity time control) unit 24a and an envelope detector unit 24b. The STC unit 24a performs correction of attenuation depending on the distance on the sound ray data formed by the reception control unit 23 in accordance with the depth of the position where the ultrasonic wave is reflected. The envelope detector unit 24b performs envelope detection processing on the sound ray data that has been corrected by the STC unit 24a so as to generate B-mode image data.

The Doppler image data generating unit 25 generates Doppler image data as image information on blood stream within the object based on frequency modulation information due to the Doppler effect in the ultrasonic echoes. The Doppler image data generating unit 25 includes a phase detector unit 25a and an MTI (moving target indicator) filter 25b. The phase detector unit 25a eliminates high frequency components from the detection signals that have been subjected to the reception focus processing based on the second ray data formed by the reception control unit 23 and performs orthogonal phase detection processing on the detection signals. The MTI filter 25b eliminates unwanted clutter components produced by the specular echo variations of a vessel wall, heart wall, or the like included in the echo signals from the detection signals that have been subjected to orthogonal phase detection processing. Thus, the Doppler image data is generated which is formed by extracting only reflection components from the blood stream.

The secondary storage unit 26 stores the B-mode data generated in the B-mode image data generating unit 24 and the Doppler image data generated in the Doppler image data generating unit 25 associated with each other.

The diagnosis support unit 27 includes a judgment criterion value setting part 27a, a judgment part 27b and a judgment criterion value storage part 27c. The criterion value setting part 27a sets a judgment criterion value for judging whether a tumor is benignant or malignant. As the judgment criterion value, as in the embodiment that will be described later, a value inputted by the operation of an operator may be set. Alternatively, a judgment criterion value corresponding to a part to be diagnosed (body mark information) or a judgment criterion value corresponding to a barcode printed in a patient's health record has been stored in the judgment criterion value storage part 27c in advance, and, when the judgment criterion value is set, the stored judgment criterion value may be read from the judgment criterion value storage part 27c and set. Further, the judgment part 27b extracts the outline of the tumor based on the B-mode image data stored in the secondary storage unit 26, calculates a degree of concentration of blood vessels in the vicinity of the tumor based on the extracted outline of the tumor, the Doppler image data corresponding to the B-mode image data and the judgment criterion value, and thereby, judges whether the tumor is benignant or malignant.

The image data generating unit 28 generates image data for displaying an ultrasonic image, a judgment result or the like based on the B-mode image data and the Doppler image data stored in the secondary storage unit 26 and the data outputted from the diagnosis support unit 27.

The tertiary storage unit 29 stores the image data generated in the image data generating unit 28. The image processing unit 30 performs various kinds of processing on the image data stored in the tertiary storage unit 29. The display unit 31 includes a display device such as a CRT and an LCD, for example, and displays an ultrasonic image based on the image data that has been subjected to image processing in the image processing unit 30.

Furthermore, the ultrasonic diagnostic apparatus according to the embodiment includes an operation unit 32, a control unit 33, an image recording unit 34, an interface 35 and a filing system 36. The operation unit 32 includes an input device such as a console 32a and a pointing device 32b, and the operation unit 32 is used when the operator inputs an instruction or information.

The control unit 33 controls the scanning control unit 11, the secondary storage unit 26 and the diagnosis support unit 27 according to the operation of the operator using the operation unit 32. For example, based on the signals inputted by the operator, the control unit 33 generates a control signal for switching modes between an imaging mode for acquiring the ultrasonic image and a diagnosis support mode for judging the tumor, and sets a judgment criterion value to be used for the judgment of the tumor. In the diagnosis support mode, the ultrasonic imaging is stopped and a still ultrasonic image and/or judgment result is displayed in the display unit 31.

Further, the control unit 33 controls the image recording unit 34 constituted by a built-in hard disk, an MO (magneto optical) etc. or the external filing system 36 via the interface 35 to record sound ray data that has been stored in the secondary storage unit 26. Furthermore, if necessary, the control unit 33 controls the secondary storage unit 26 to store sound ray data that has been recorded in the image recording unit 34 or the external filing system 36 or the image data that has been generated by an external modality such as X-ray imaging equipment.

The above-described diagnosis support unit 27 and control unit 33 can be realized by a CPU and software (program). As a recording medium for recording software (program), other than a built-in hard disk, a flexible disk, an MO, an MT, a RAM, a CD-ROM, a DVD-ROM, or the like can be used.

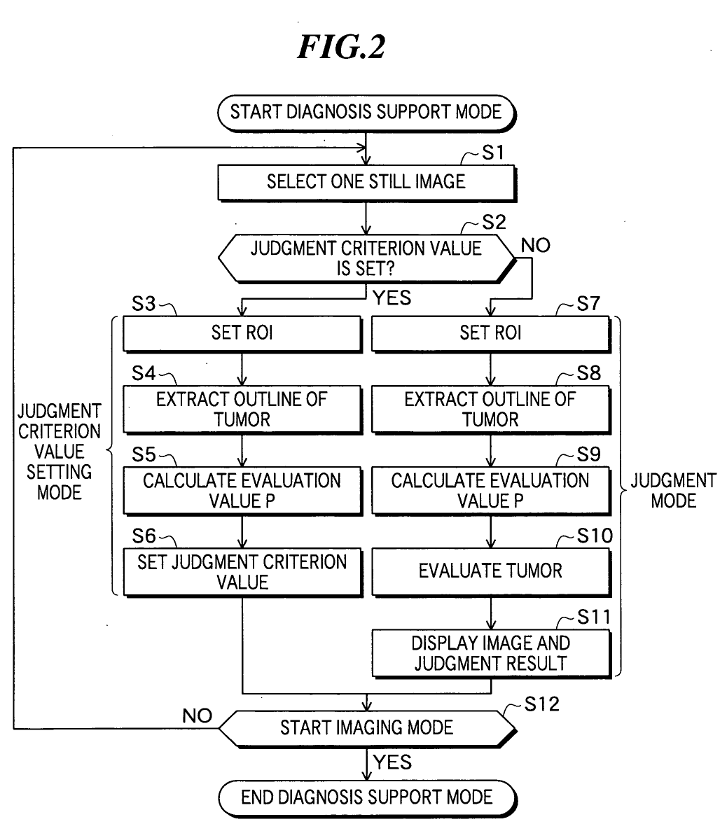

Next, the operation of the ultrasonic diagnosing apparatus according to the embodiment will be described by referring to FIGS. 2-10. FIG. 2 is a flowchart showing the operation of the ultrasonic diagnosing apparatus according to the embodiment in the diagnosis support mode. In the embodiment, the diagnosis support is performed by judging the property of the tumor (benignant or malignant) based on the degree of concentration of the blood stream in a tissue within the object.

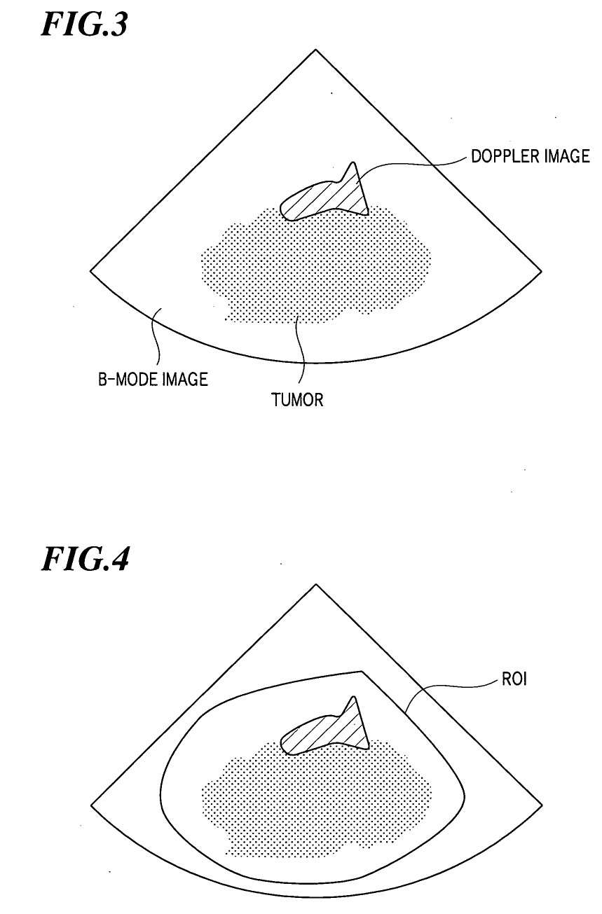

When the operator operates the operation unit 32 to switch from the imaging mode to the diagnosis support mode, a still image corresponding to the B-mode image data and the Doppler image data is displayed on the display unit 31. The image displayed here is an image formed by synthesizing the B-mode image and the Doppler image as shown in FIG. 3. In FIG. 3, the shaded area represents the Doppler image and the area except the shaded area within the sector represents the B-mode image. Further, the area shown by dots within the B-mode image represents the tumor.

Alternatively, as an image to be displayed on the display unit 31, an image recorded in the image recording unit 34 or the filing system 36 may be used. In this case, in place of the combination of the B-mode image and the Doppler image, a combination of an X-ray image and the Doppler image, a CT image and the Doppler image, or an MRI image and the Doppler image can be used. In that case, it is necessary to have recorded an image set, in which the image generated by each modality and the Doppler image are aligned in advance, in the filing system 36.

First, at step S1 in FIG. 2, the operator selects one still image from plural still images displayed based on the B-mode image data and the Doppler image data stored in the secondary storage unit 26. At that time, it is desired to select the image in which the position of the tumor and the property of the tumor (benignant or malignant) are clearly known.

Then, at step S2, the diagnosis support unit 27 judges whether the judgment criterion value to be used for the judgment of the tumor has been set or not. In the case where the judgment criterion value has not been set, the judgment criterion value is set in the judgment criterion value setting mode. On the other hand, in the case where the judgment criterion value has been set, the property of the tumor is judged in the judgment mode.

Steps S3 to S6 show the operation of the ultrasonic diagnosing apparatus in the judgment criterion value setting mode.

At step S3, the operator confirms the still image being displayed on the display unit 31, and sets ROI (region of interest) by using the pointing device 32b as shown in FIG. 4. The information on ROI that has been set by the operator is inputted to the judgment criterion value setting part 27a of the diagnosis support unit 27 via the control unit 33.

Then, at step S4, the judgment criterion value setting part 27a extracts the outline of the tumor based on the B-mode image data including the image of the tumor as shown in FIG. 5.

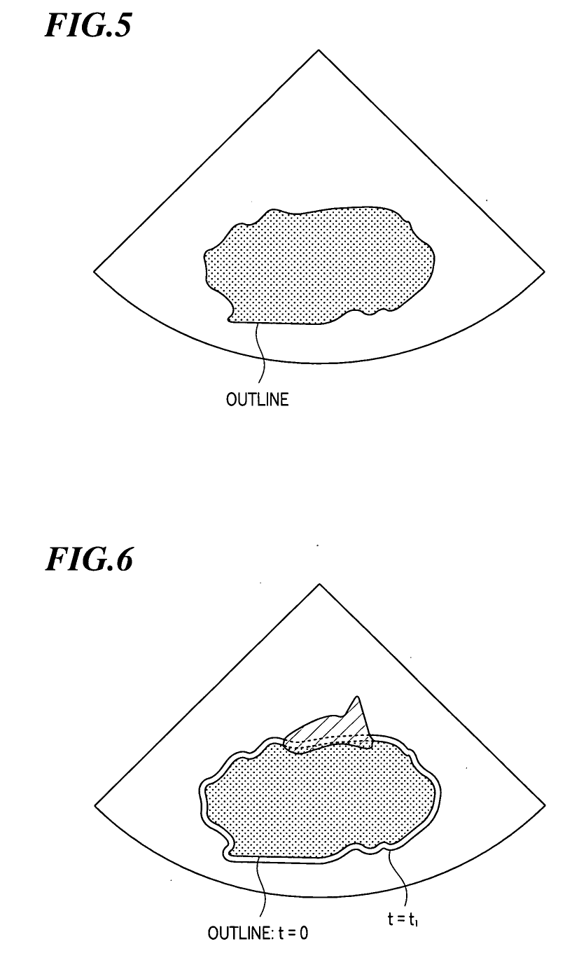

At step S5, the judgment criterion value setting part 27a sets as a measurement area an area surrounded by a line apart from the outline that has been extracted at step S4 by a distance “t” as shown in FIG. 6, and calculates an evaluation value “P” expressed by the following equation (1) based on the distance “t”.
P═S_{D}/S_{A} (1)
Where, “S_{D}” is a value obtained by weighting the area of the measurement area at the distance “t” from the outline by using the brightness value in the Doppler image. This value “S_{D}” can be obtained by integrating the unit area while weighting it by Doppler intensity. On the other hand, “S_{A}” is an area of the measurement area at the distance “t” from the outline. In FIG. 6, the measurement area in the case where t=t₁ is shown.

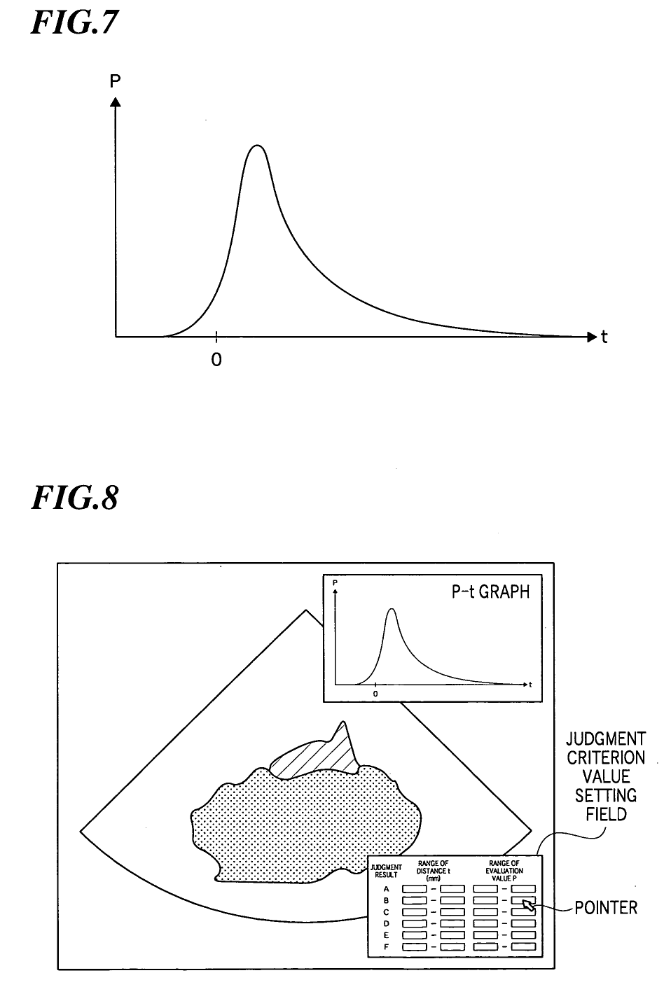

FIG. 7 shows the relationship between the evaluation value “P” and the distance “t”. Here, since the Doppler image also exists at inner side than the outline (t=0), there is a range in which P>0 is held even if t<0. Furthermore, the judgment criterion value setting part 27a outputs data to the image data generating unit 28, and thereby, as shown in FIG. 8, synthesizes a graph representing the relationship between the evaluation value “P” and the distance “t” and a judgment criterion value setting field with the ultrasonic image to display it on the display unit 31.

At step S6, the operator inputs numerical values of the range of the distance “t” and the range of the evaluation value “P” in the judgment criterion value setting field by using the console 32a or the like while referring to the graph representing the relationship between the evaluation value “P” and the distance “t” being displayed on the display unit 31. At that time, for example, numerical values may be inputted from the following point of view. In a malignant tumor, it is considered that nutrient vessels develop and a large amount of blood flows into the tumor. Accordingly, in the vicinity of the outline of the tumor (near t=0), the case where the range of the Doppler image representing the blood stream and the brightness value thereof are large, that is, the case where the evaluation value “P” is large is associated with the judgment result “A” which means it is highly possible that the tumor is malignant, for example.

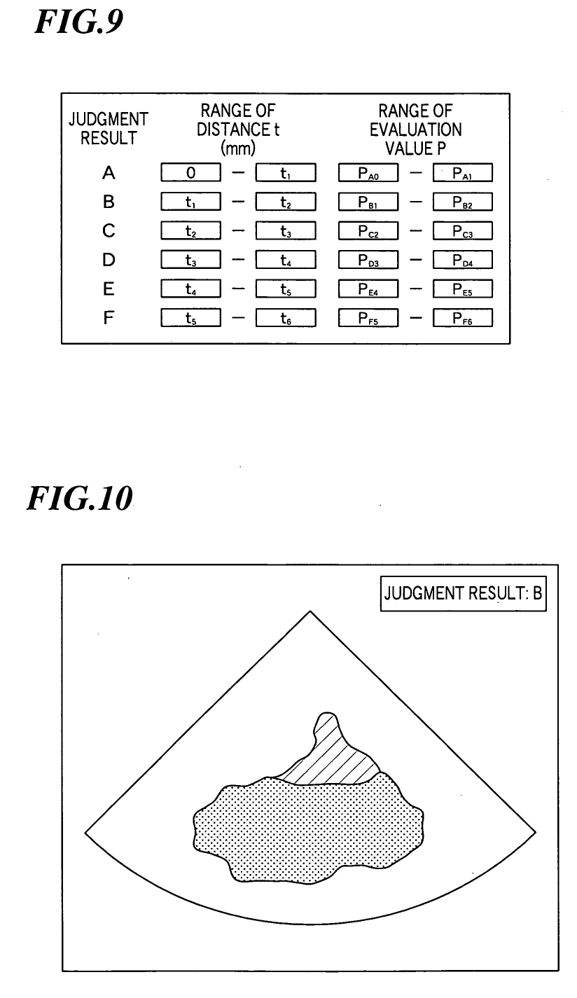

As shown in FIG. 9, the range of the distance “t” and the range of the evaluation value “P”, which have been set, are set as criterion values corresponding to the judgment results “A”, “B”, . . . to be shown in the judgment mode.

When the operator sets the range of the judgment criterion value, the set judgment criterion value is stored in correspondence with the B-mode image data and the Doppler image data in the secondary storage unit 26 under the control of the judgment criterion value setting part 27a. Further, in the case where the B-mode image data and the Doppler image data are recorded in the image recording unit 34 or the filing system 36, the set judgment criterion value is recorded as headers of the B-mode image data and the Doppler image data under the control of the control unit 33.

Next, steps S7 to S11 show the operation of the ultrasonic diagnosing apparatus in the judgment mode.

At step S7, the operator confirms the image being displayed on the display unit 31 and sets ROI by using the pointing device 32b with respect to the image as a target of judgment (synthesized image of the B-mode image and the Doppler image). The information on ROI that has been set by the operator is inputted to the judgment part 27b of the diagnosis support unit 27 via the control unit 33.

At step S8, the judgment part 27b extracts the outline of the tumor based on the displayed B-mode image data. Then, at step S9, the judgment part 27b sets the measurement area “t” corresponding to the judgment results “A”, “B”, . . . that have been set in the evaluation criterion setting mode based on the outline that has been extracted at step S8, and calculates the evaluation value “P” by using the equation (1). Furthermore, at steps 10, the judgment part 27b compares the evaluation value “P” that has been calculated at step S9 with the judgment criterion value that has been set in advance, and judges whether the evaluation value “P” falls within the range of the judgment criterion values corresponding to the judgment results “A”, “B”, . . . or not.

At step S11, the judgment part 27b outputs data representing a judgment result to the image data generating unit 28, and thereby, as shown in FIG. 10, synthesizes the judgment result with the B-mode image and the Doppler image to display it on the display unit 31. Further, this judgment result is outputted to the secondary storage unit 26 and stored as headers of the corresponding B-mode image data and the Doppler image data. Furthermore, in the case where the B-mode image data and the Doppler image data are recorded in the image recording unit 34 or the filing system 36, the judgment result is recorded as headers of the B-mode image data and the Doppler image data under the control of the control unit 33.

After the setting of the judgment criterion value in the judgment criterion value setting mode or the judgment with respect to one still image in the judgment mode is completed, at step S12, judgment as to whether the imaging mode is started or not is performed. In the case where the imaging mode is started again, the diagnosis support mode is ended and the imaging mode is started, while, in the case where the imaging mode is not started, the processing moves to step S1 and the diagnosis support is continued.

A doctor performs medical diagnosis based on the results “A”, “B”, . . . that have been thus judged with respect to the image as a target of judgment. As a diagnostic procedure at that time, from the relationship with the set range of the judgment criterion value, for example, a tumor may be diagnosed as being malignant if the result corresponds to the judgment result “A” or “B”, or if the result corresponds to two or more of the judgment results “A” to “C”.

In the embodiment, the area of the blood stream part is obtained based on the Doppler image, however, the area of the blood stream part can be obtained by the contrast echo method using a contrast agent. In this case, the area of the blood stream part can be obtained based on the difference image between an ultrasonic image before injection of the contrast agent and an ultrasonic image after the injection of the contrast agent.

Furthermore, in the embodiment, by using the Doppler image, whether the tumor is benignant or malignant is judged based on the degree of concentration of the blood vessels in the tissue within the object, however, in addition to this, or in place of this, the complexity of the outline in the tissue within the object may be used as a judgment criterion. The complexity of the outline can be calculated based on, for example, the ratio of the length of the outline to the length of the circumference of an ellipse as an approximation of the outline.

1. An ultrasonic diagnosing apparatus comprising:
image information generating means for generating image information on an object to be inspected based on a plurality of ultrasonic echoes obtained by scanning the object while sequentially transmitting a plurality of ultrasonic beams from an ultrasonic probe;
image information storage means for storing the image information generated by said image information generating means;
judgment criterion value setting means for setting as a judgment criterion value at least one kind of value to be used for judging at least one kind of evaluation value on a tissue within the object with respect to the image information stored in said image information storage means;
judging means for judging a state of said tissue by comparing the at least one kind of evaluation value on said tissue with the set judgment criterion value with respect to the image information as a target of judgment stored in said image information storage means; and
display means for displaying an ultrasonic image based on the image information stored in said-image information storage means in an imaging mode, and displaying a judgment result by said judging means in a diagnosis support mode.

2. The ultrasonic diagnosing apparatus according to claim 1, wherein:
said image information generating means includes first image information generating means for generating image information on a tissue within the object based on intensity of the plurality of ultrasonic echoes and second image information generating means for generating image information on blood stream within the object based on frequency modulation information due to Doppler effect in the plurality of ultrasonic echoes; and
said display means displays an ultrasonic image based on the image information generated by said first image information generating means in the imaging mode, and displays an ultrasonic image based on the image information generated by said first and second image information generating means together with the judgment result by said judging means in the diagnosis support mode.

3. The ultrasonic diagnosing apparatus according to claim 1, wherein:
said judgment criterion value setting means sets as a judgment criterion value at least one kind of value to be used for judging at least one kind of evaluation value on a tissue within the object calculated by extracting an outline of the tissue with respect to the image information as a target of judgment stored in said image information storage means; and
said judging means judges a state of the tissue within the object by extracting the outline of said tissue based on the image information stored in said image information storage means, calculating at least one kind of evaluation value on said tissue based on the extracted outline, and comparing the calculated at least one kind of evaluation value with the set judgment criterion value to cause said display means to display a judgment result.

4. The ultrasonic diagnosing apparatus according to claim 2, wherein:
said judgment criterion value setting means sets as a judgment criterion value at least one kind of value to be used for judging at least one kind of evaluation value on a tissue within the object calculated by extracting an outline of the tissue with respect to the image information as a target of judgment stored in said image information storage means; and
said judging means judges a state of the tissue within the object by extracting the outline of said tissue based on the image information stored in said image information storage means, calculating at least one kind of evaluation value on said tissue based on the extracted outline, and comparing the calculated at least one kind of evaluation value with the set judgment criterion value to cause said display means to display a judgment result.

5. The ultrasonic diagnosing apparatus according to claim 2, wherein said judging means judges a state of the tissue within the object by comparing an evaluation value on a degree of concentration of the blood stream in the tissue within the object with a plurality of criterion values with respect to image information generated by said first and second image information generating means.

6. The ultrasonic diagnosing apparatus according to claim 3, wherein said judging means judges a state of the tissue within the object by comparing an evaluation value on complexity of the outline in the tissue within the object with a plurality of criterion values with respect to image information generated by said image information generating means.

7. The ultrasonic diagnosing apparatus according to claim 4, wherein said judging means judges a state of the tissue within the object by comparing an evaluation value on a degree of concentration of the blood stream in the tissue within the object with a first group of criterion values with respect to image information generated by said first and second image information generating means and comparing an evaluation value on complexity of the outline in the tissue within the object with a second group of criterion values with respect to image information generated by said first image information generating means.

8. The ultrasonic diagnosing apparatus according to claim 5, wherein said evaluation value on the degree of concentration of the blood stream in the tissue within the object is obtained by dividing an area weighted by a brightness value in the image of said tissue obtained by said second image information generating means by an area of said tissue in the image obtained by said first image information generating means.

9. The ultrasonic diagnosing apparatus according to claim 7, wherein said evaluation value on the degree of concentration of the blood stream in the tissue within the object is obtained by dividing an area weighted by a brightness value in the image of said tissue obtained by said second image information generating means by an area of said tissue in the image obtained by said first image information generating means.

10. The ultrasonic diagnosing apparatus according to claim 6, wherein said evaluation value on complexity of the outline in the tissue within the object is obtained by dividing a length of the outline of said tissue by a length of circumference of an ellipse as an approximation of the outline.

11. The ultrasonic diagnosing apparatus according to claim 7, wherein said evaluation value on complexity of the outline in the tissue within the object is obtained by dividing a length of the outline of said tissue by a length of circumference of an ellipse as an approximation of the outline.

12. The ultrasonic diagnosing apparatus according to claim 9, wherein said evaluation value on complexity of the outline in the tissue within the object is obtained by dividing a length of the outline of said tissue by a length of circumference of an ellipse as an approximation of the outline.

13. The ultrasonic diagnosing apparatus according to claim 1, further comprising:
second storage means for storing at least one kind of value to be used for judging at least one kind of evaluation value on a tissue within the object;
wherein said judgment criterion value setting means sets the value stored in said second storage means as the judgment criterion value.

14. The ultrasonic diagnosing apparatus according to claim 1, further comprising:
an interface for connecting said ultrasonic diagnosing apparatus to an external filing system; and
control means for reading out image data recorded in said filing system via said interface and controlling said image information storage means to store the image data.

15. The ultrasonic diagnosing apparatus according to claim 14, wherein said image information storage means stores (i) image information on one of an X-ray image, a CT image and an MRI image and (ii) image information generated based on frequency modulation information due to Doppler effect in the plurality of ultrasonic echoes.

16. The ultrasonic diagnosing apparatus according to claim 1, wherein said image information storage means stores said at least one kind of judgment criterion value and said at least one kind of evaluation value as headers of the image information.

17. The ultrasonic diagnosing apparatus according to claim 1, wherein said judgment criterion value setting means and said judging means are formed as functional blocks realized by a CPU and software.
